(19) United States
(12) Patent Application Publication
(10) Pub. No.: US 2025/0281144 A1
(43) Pub. Date: Sep. 11, 2025

(54) PCCT APPARATUS AND CONTROL METHOD THEREOF

(71) Applicant: FUJIFILM Corporation, Tokyo (JP)

(72) Inventors: Fuyuhiko TERAMOTO, Tokyo (JP); Shinichi KOJIMA, Tokyo (JP); Kazuma YOKOI, Tokyo (JP)

(73) Assignee: FUJIFILM Corporation, Tokyo (JP)

(21) Appl. No.: 19/019,496

(22) Filed: Jan. 14, 2025

(30) Foreign Application Priority Data
Mar. 5, 2024 (JP) 2024-033095

(51) Int. Cl. A61B 6/58; A61B 6/00; A61B 6/03; A61B 6/42; G06T 11/00

(52) U.S. Cl. CPC A61B 6/584; A61B 6/032; A61B 6/4241; G06T 11/006; A61B 6/484

(57) ABSTRACT

Provided are a PCCT apparatus and a control method thereof capable of improving correction accuracy of a projection value even in a range in which the projection value is smaller.

A PCCT apparatus includes a scanner that rotates an X-ray source which irradiates a subject with X-rays and a photon counting detector which detects X-rays transmitted through the subject for each of a plurality of energy bins, around the subject, an image generation unit that generates a tomographic image by using projection data calculated based on an output of the photon counting detector, and a controller that controls each unit, in which the controller calculates a conversion expression for converting a projection value of the subject into a transmission length by using a plurality of projection values acquired by performing rotational imaging on a phantom which is disposed not to overlap a rotation center of the scanner and of which a material, a shape, and a size are known, along with a plurality of transmission lengths of the phantom.

### CLAIM OF PRIORITY

The present application claims priority from Japanese Patent Application JP 2024-033095 filed on Mar. 5, 2024, the content of which is hereby incorporated by reference into this application.

### BACKGROUND OF THE INVENTION

### 1. Field of the Invention

The present invention relates to a photon counting computed tomography (PCCT) apparatus that generates a tomographic image of a subject by using projection data divided into a plurality of energy bins, and particularly relates to a technique of correcting non-linearity of a projection value.

### 2. Description of the Related Art

An X-ray CT apparatus uses projection data from multiple directions that is obtained by rotating, around a subject, an X-ray source that irradiates a subject with X-rays and an X-ray detector that detects the X-rays transmitted through the subject to generate a tomographic image of the subject. The generated tomographic image depicts an organ shape of the subject and is used for image diagnosis.

Since an X-ray absorption coefficient of substances included in the subject is generally smaller as an X-ray energy is higher, in a case where the emitted X-rays have a predetermined energy width, so-called beam hardening, in which the average energy of the X-rays is increased by the transmission of the X-rays through the subject, occurs. Since the beam hardening deteriorates the image quality of the tomographic image by making a relationship between a projection value obtained from an X-ray dose transmitted through the subject and a transmission length of the subject non-linear, correction for the projection value is required.

JP2004-180808A discloses that a projection value is corrected by using a correction function obtained by performing beam hardening correction and a plurality of function fittings on a sinogram obtained by performing rotational imaging on a phantom disposed to be shifted from a center of an imaging region.

### SUMMARY OF THE INVENTION

However, in JP2004-180808A, consideration for correction accuracy in a range in which the projection value is smaller, that is, in a range in which the transmitted X-ray dose is larger is insufficient. In a PCCT apparatus including a photon counting detector, which counts incident X-ray photons one by one, as an X-ray detector, a proportional relationship between input and output is broken due to an influence of a pile-up or the like that occurs in a case where a larger number of X-ray photons are detected, that is, in a case where the X-ray dose is larger, and thus the correction accuracy of the projection value is decreased. The decrease in the correction accuracy of the projection value deteriorates the image quality of the tomographic image.

Therefore, an object of the present invention is to provide a PCCT apparatus and a control method thereof capable of improving correction accuracy of a projection value even in a range in which the projection value is smaller.

In order to achieve the above object, a PCCT apparatus according to an aspect of the present invention comprises: a scanner that rotates an X-ray source which irradiates a subject with X-rays and a photon counting detector which detects X-rays transmitted through the subject for each of a plurality of energy bins, around the subject; an image generation unit that generates a tomographic image by using projection data calculated based on an output of the photon counting detector; and a controller that controls each unit, in which the controller calculates a conversion expression for converting a projection value of the subject into a transmission length by using a plurality of projection values acquired by performing rotational imaging on a phantom which is disposed not to overlap a rotation center of the scanner and of which a material, a shape, and a size are known, along with a plurality of transmission lengths of the phantom.

In addition, in the present invention, there is provided a control method of a PCCT apparatus including a scanner that rotates an X-ray source which irradiates a subject with X-rays and a photon counting detector which detects X-rays transmitted through the subject for each of a plurality of energy bins, around the subject, and an image generation unit that generates a tomographic image by using projection data calculated based on an output of the photon counting detector, the control method comprising: calculating a conversion expression for converting a projection value of the subject into a transmission length by using a plurality of projection values acquired by performing rotational imaging on a phantom which is disposed not to overlap a rotation center of the scanner and of which a material, a shape, and a size are known, along with a plurality of transmission lengths of the phantom.

According to the present invention, it is possible to provide a PCCT apparatus and a control method thereof capable of improving correction accuracy of a projection value even in a range in which the projection value is smaller.

### BRIEF DESCRIPTION OF THE DRAWINGS

FIG. 1 is a diagram showing an overall configuration of a PCCT apparatus.

FIG. 2 is a graph showing an example of X-rays divided into a plurality of energy bins.

FIG. 3 is a flowchart showing an example of a processing flow in Example 1.

FIG. 4 is a diagram showing an example of a projection value of a phantom that is disposed not to overlap a rotation center of a scanner.

FIG. 5 is a diagram illustrating a jig that supports a phantom.

FIGS. 6A and 6B are diagrams illustrating calculation of a position of a phantom based on projection data.

FIG. 7 is a diagram illustrating calculation of a position of a phantom based on a tomographic image generated by using projection data.

FIG. 8 is a diagram illustrating calculation of transmission lengths of a phantom.

FIG. 9 is a graph illustrating calculation of an expression for converting a projection value into a transmission length.

FIGS. 10A to 10D are diagrams illustrating a case where the phantom is disposed at a rotation center of the scanner.

FIGS. 11A to 11D are diagrams illustrating a case where a phantom is disposed on a top plate.

FIGS. 12A to 12D are diagrams illustrating a case where a phantom having a polygonal cross-sectional shape is used.

### DESCRIPTION OF THE PREFERRED EMBODIMENTS

Hereinafter, examples of a photon counting computed tomography (PCCT) apparatus according to an embodiment of the present invention will be described with reference to the accompanying drawings. In the following description and the accompanying drawings, components having the same functional configuration will be denoted by the same reference numerals, and duplicated descriptions will not be repeated.

### Example 1

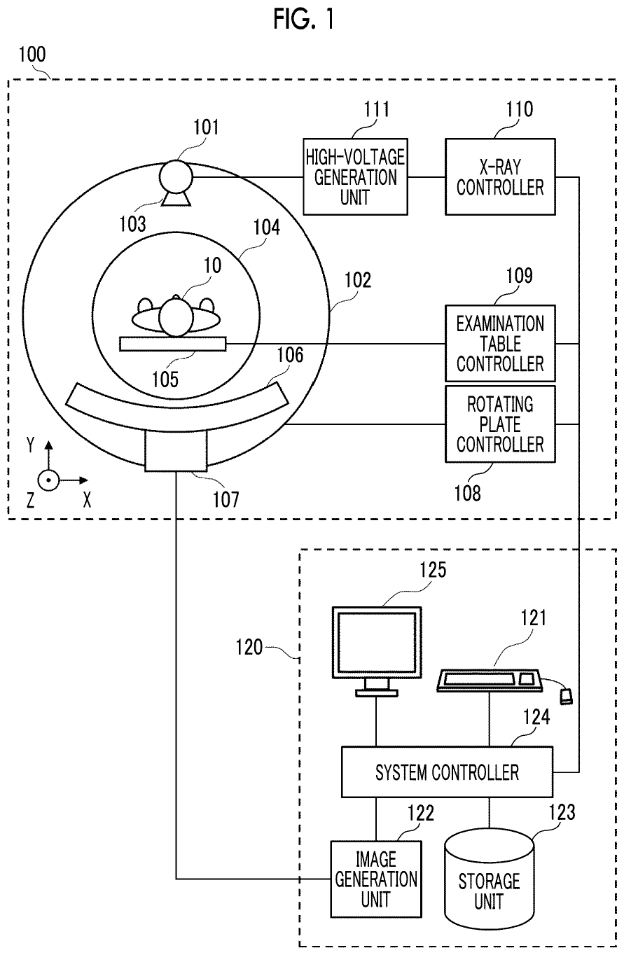

An overall configuration of a PCCT apparatus in Example 1 will be described with reference to FIG. 1. The PCCT apparatus includes a scanner 100 and an operation unit 120. The scanner 100 is installed in an imaging room surrounded by a shielding material that blocks X-rays, and the operation unit 120 is installed in an operation room located outside the imaging room. A rotation axis direction of the scanner 100 is referred to as a Z-axis, a horizontal direction perpendicular to the Z-axis is referred to as an X-axis, and a vertical direction perpendicular to the Z-axis is referred to as a Y-axis.

The scanner 100 includes an X-ray source 101, a rotating plate 102, a collimator 103, a photon counting detector 106, a data collection unit 107, an examination table 105, a rotating plate controller 108, an examination table controller 109, an X-ray controller 110, and a high-voltage generation unit 111. The X-ray source 101 is a device that irradiates a subject 10 with X-rays placed on the examination table 105 and is, for example, an X-ray tube device. The collimator 103 is a device that limits an irradiation range of X-rays. The rotating plate 102 includes an opening portion 104 through which the subject 10 placed on the examination table 105 enters, is equipped with the X-ray source 101 and the photon counting detector 106, and rotates the X-ray source 101 and the photon counting detector 106, around the subject 10.

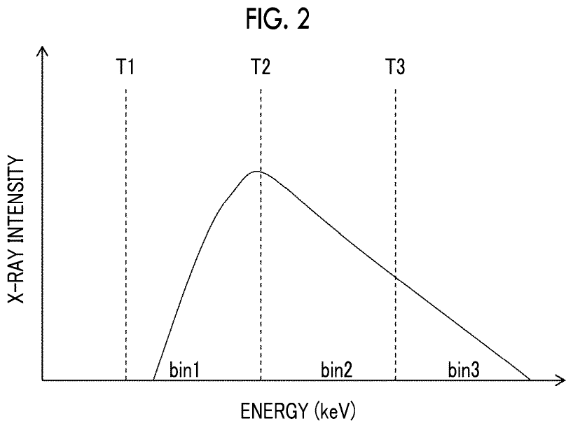

The photon counting detector 106 is a device that is disposed to face the X-ray source 101 and acquires a spatial distribution of X-rays by including a plurality of detection elements that detect the X-rays. The detection elements of the photon counting detector 106 are arranged two-dimensionally in a rotation direction and a rotation axis direction of the rotating plate 102, detect incident X-ray photons one by one, and discriminate each X-ray photon into the plurality of energy bins. FIG. 2 shows the detected X-rays divided into three energy bins which are respectively from T1 to T2, from T2 to T3, and from T3 as bin1, bin2, and bin3. The data collection unit 107 is a device that collects the spatial distribution of the X-rays for each energy bin acquired by the photon counting detector 106 as digital data.

The rotating plate controller 108 is a device that controls rotation and inclination of the rotating plate 102. The examination table controller 109 is a device that controls upward, downward, forward, backward, leftward, and rightward movements of the examination table 105. The high-voltage generation unit 111 is a power source that generates a tube voltage, which is a voltage applied to the X-ray source 101, and a tube current, which is a current supplied to the X-ray source 101. The X-ray controller 110 is a device that controls an output of the high-voltage generation unit 111. The rotating plate controller 108, the examination table controller 109, and the X-ray controller 110 are, for example, a micro-processing unit (MPU) or the like.

The operation unit 120 includes an input unit 121, an image generation unit 122, a display unit 125, a storage unit 123, and a system controller 124. The input unit 121 is a device that is used to input examination data such as a name of the subject 10, an examination date and time, and an imaging condition, and is, for example, a keyboard, a pointing device, a touch panel, or the like. The image generation unit 122 is a device that generates a tomographic image by using digital data collected by the data collection unit 107, and is, for example, an MPU, a graphics processing unit (GPU), or the like. The display unit 125 is a device that displays the tomographic image or the like generated by the image generation unit 122, and is, for example, a liquid crystal display, a touch panel, or the like. The storage unit 123 is a device that stores the digital data collected by the data collection unit 107, the tomographic image generated by the image generation unit 122, a program to be executed by the system controller 124, data to be used by the program, and the like, and is, for example, a hard disk drive (HDD), a solid state drive (SSD), or the like. The system controller 124 is a device that controls each unit such as the rotating plate controller 108, the examination table controller 109, and the X-ray controller 110, and is, for example, a central processing unit (CPU).

With the generation of the tube voltage and the tube current by the high-voltage generation unit 111 based on the imaging condition set via the input unit 121, the X-rays according to the imaging condition are emitted from the X-ray source 101 to the subject 10. The photon counting detector 106 detects the X-rays emitted from the X-ray source 101 and transmitted through the subject 10 with multiple detection elements to acquire the spatial distribution of the transmitted X-rays. The rotating plate 102 is controlled by the rotating plate controller 108 and rotates based on the imaging condition input through the input unit 121, particularly a rotation speed or the like. The examination table 105 is controlled by the examination table controller 109 and moves relative to the rotating plate 102 to move an imaging position designated with respect to the subject 10 to an imaging field of view, which is a range in which the transmitted X-rays are detected.

With repetition of the emission of the X-rays by the X-ray source 101 and the detection of the X-rays by the photon counting detector 106 together with the rotation of the rotating plate 102, projection data of the subject 10 is measured at various projection angles. In the projection data, a view representing each projection angle is associated with a channel (ch) number and a column number which are detection element numbers of the photon counting detector 106. The measured projection data is transmitted to the image generation unit 122. The image generation unit 122 performs back-projection processing on a plurality of projection data to generate the tomographic image. The generated tomographic image is displayed on the display unit 125 or stored in the storage unit 123 as a medical image.

Since the X-ray absorption coefficient of substances included in the subject 10 is generally smaller as the X-ray energy is higher, in a case where the X-rays emitted from the X-ray source 101 have a predetermined energy width, so-called beam hardening occurs in the X-rays transmitted through the subject 10. Since the beam hardening makes a relationship between the projection value obtained from the X-ray dose transmitted through the subject 10 and the transmission length of the subject 10 non-linear, correction for the projection value of the subject 10 is required. In particular, in the PCCT apparatus including the photon counting detector 106, the correction accuracy in a range in which the projection value is smaller is important because the projection value is further non-linear due to the influence of the pile-up or the like, which occurs in a case where the X-ray dose is larger, in addition to the beam hardening. Therefore, in Example 1, even in a range in which the projection value is smaller, a conversion expression that can convert the projection value of the subject 10 into the transmission length with high accuracy is calculated.

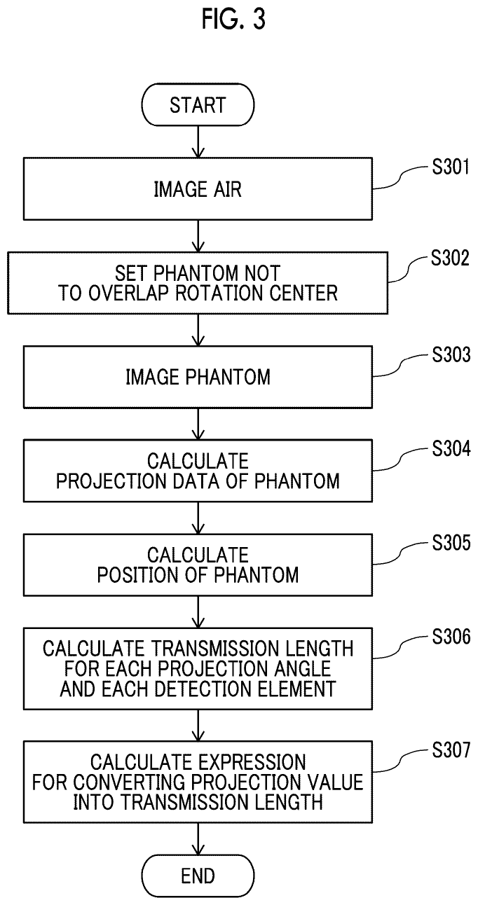

An example of a processing flow in Example I will be described step by step with reference to FIG. 3.

### S301

The system controller 124 operates the scanner 100 in a state where nothing is placed in the opening portion 104 to image air. The imaging data of the air is used, for example, to calculate the sensitivity of each detection element of the photon counting detector 106.

### S302

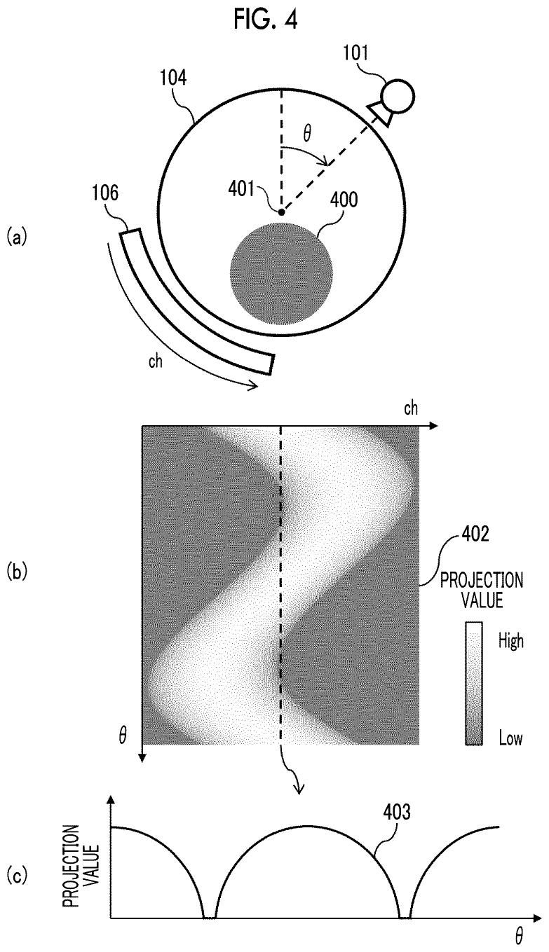

As shown in (a) of FIG. 4, the phantom 400 is set in the opening portion 104 not to overlap the rotation center 401 of the scanner 100. The phantom 400 has a known material, shape, and size. (a) of FIG. 4 illustrates the phantom 400 in which a cross-sectional shape of the phantom 400 is a circle and a diameter of the circle is less than a radius of the opening portion 104.

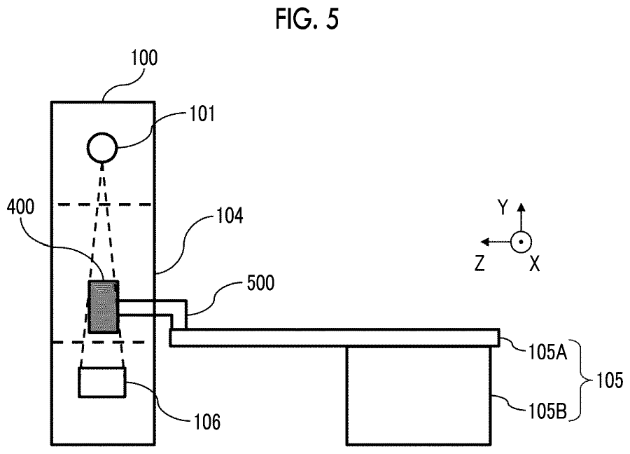

The phantom 400 is supported from the rotation axis direction of the scanner 100 by, for example, a jig 500 shown in FIG. 5. The jig 500 illustrated in FIG. 5 is attached to a distal end of the top plate 105A on the examination table 105. The phantom 400 is set in the opening portion 104 by sliding the top plate 105A in the rotation axis direction of the scanner 100. In addition, a position of the phantom 400 is adjusted by upward, downward, leftward, and rightward movements of the base 105B that supports the top plate 105A from below.

### S303

The system controller 124 operates the scanner 100 in a state in which the phantom 400 is set in the opening portion 104 to perform rotational imaging on the phantom 400.

### S304

The system controller 124 calculates projection data of the phantom 400 by using imaging data of the air obtained in S301 and imaging data of the phantom 400 obtained in S303. Prior to calculating the projection data, pre-processing such as offset correction, logarithmic conversion processing, and reference correction may be performed on each piece of imaging data.

(b) of FIG. 4 illustrates projection data 402 of the phantom 400 set not to overlap the rotation center 401. The projection data 402 indicates a projection value for each coordinate represented by a projection angle θ and a channel number ch of the detection element. In addition, (c) of FIG. 4 illustrates a projection profile 403 representing a projection value for each projection angle θ in a center ch. In the projection data 402 of the phantom 400 set not to overlap the rotation center 401, the projection profile 403 of most of the ch includes a projection value from zero to the maximum value.

### S305

The system controller 124 calculates the position of the phantom 400. For example, the position of the phantom 400 is calculated based on the amount of upward, downward, leftward, and rightward movement of the base 105B of the examination table 105. In addition, the position of the phantom 400 may be calculated based on the projection data 402 calculated in S304.

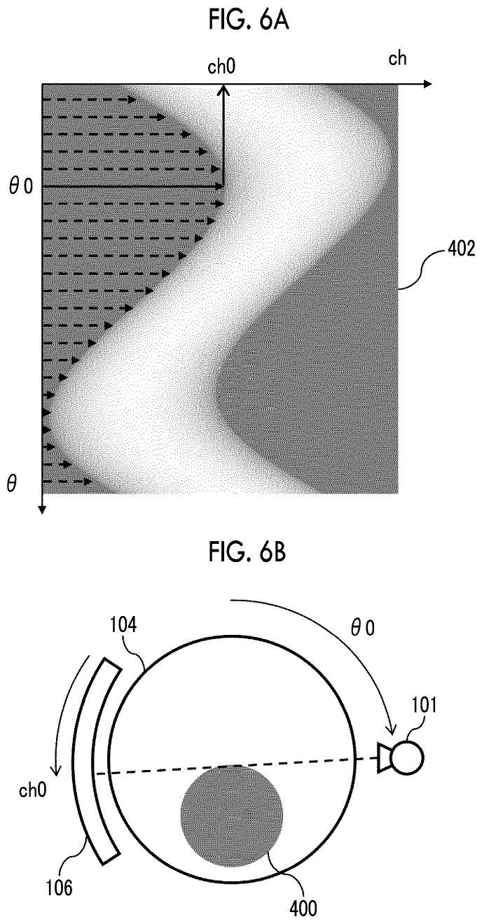

An example of the calculation of the position of the phantom 400 based on the projection data 402 will be described with reference to FIGS. 6A and 6B. First, at each projection angle θ of the projection data 402, a range of ch, which is a projection value of the air, is extracted. Then, the position of the phantom 400 is calculated by specifying the projection angle at which the range of ch, which is the projection value of the air, is widest, along with the ch that is the boundary of the projection value of the air at the projection angle. In FIG. 6A, the range of ch, which is the projection value of the air, is indicated by arrows, and it is indicated that the projection angle at which the extracted range of ch is widest is θ0, and the boundary of the projection value of the air at the projection angle θ0 is ch0. Since the shape and size of the phantom 400 are known, the position of the phantom 400 is calculated based on the specified θ0 and ch0.

In addition, the position of the phantom 400 may be calculated by fitting the boundary of the projection value of the air in the projection data 402 of the phantom 400 with a sine curve. With the calculation of the position of the phantom 400 based on the projection data 402, the position accuracy is improved as compared with the position of the phantom 400 calculated based on the movement amount of the base 105B. In addition, the position of the phantom 400 may be calculated based on the tomographic image generated by using the projection data 402.

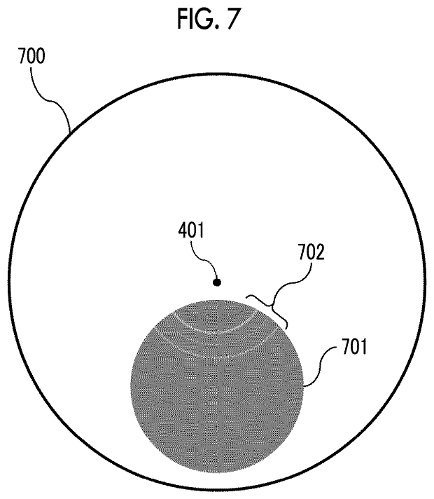

An example of the calculation of the position of the phantom 400 based on the tomographic image generated by using the projection data 402 will be described with reference to FIG. 7. FIG. 7 illustrates an image in which a phantom region 701 is included in an imaging field of view 700 as the tomographic image generated by using the projection data 402. Since the non-linearity of the projection data 402 is not corrected, the phantom region 701 includes the ring artifact 702. However, since the boundary of the phantom region 701 can be specified, the position of the phantom 400 can be calculated. In addition, with the calculation of the position of the phantom 400 based on the tomographic image, the position accuracy is improved as compared with the position of the phantom 400 calculated based on the movement amount of the base 105B. Return to the description of FIG. 3.

### S306

The system controller 124 calculates the transmission length of the phantom 400 at the position calculated in S305 for each projection angle θ and each channel number ch of the detection element.

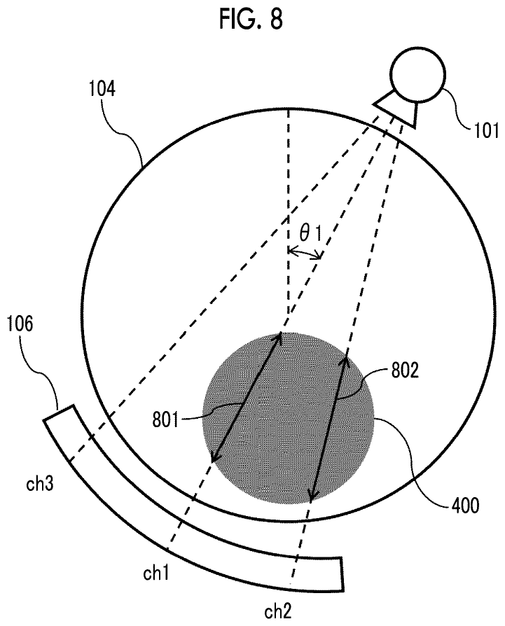

The calculation of the transmission length of the phantom 400 will be described with reference to FIG. 8. Since the position of the phantom 400 having a known shape and size is required, a length of a portion where a straight line connecting the X-ray source 101 and each detection element of the photon counting detector 106 overlaps the phantom 400 is calculated as the transmission length of the phantom 400. FIG. 8 shows a transmission length 801 in the detection element of ch1 and a transmission length 802 in the detection element of ch2 in a case where the projection angle is θ1. The transmission length in the detection element of ch3 is zero.

### S307

The system controller 124 calculates a conversion expression for converting the projection value of the subject 10 into the transmission length by using the projection data 402 calculated in S304 and the transmission length calculated in S305.

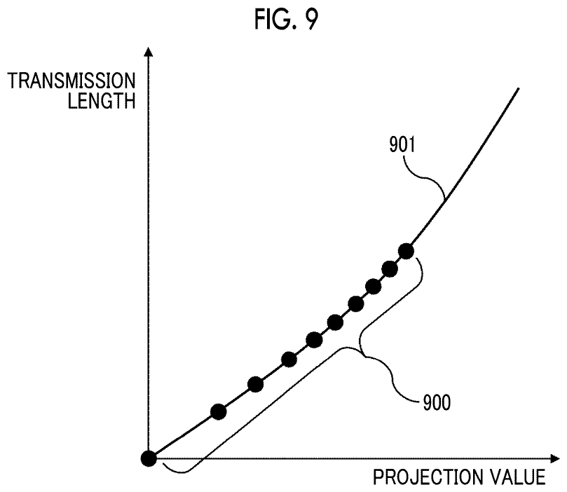

The calculation of the conversion expression will be described with reference to FIG. 9. The transmission length calculated in S305 and the projection value obtained from the projection data 402 are obtained for each projection angle θ and each channel number ch of the detection element, and thus the obtained transmission length and projection value are associated with each other by the projection angle θ and the channel number ch. Therefore, as illustrated in FIG. 9, a set of the transmission length and the projection value associated with each other by the projection angle θ and the channel number ch is plotted as one measurement point 900 on a coordinate plane in which the projection value is on a horizontal axis and the transmission length is on a vertical axis. FIG. 9 is a graph in which each of measurement points 900 for each of a plurality of projection angles θ are plotted at a predetermined channel number ch. That is, each of the measurement points 900 shown in FIG. 9 is a set of a transmission length and a projection value obtained for each of different projection angles θ at the same channel number ch.

Then, a conversion expression for converting the projection value into the transmission length at a predetermined channel number ch is calculated by fitting a plurality of the measurement points 900 illustrated in FIG. 9 with an approximate curve 901 represented by a high-order function or the like. The calculated conversion expression is used in a case where the projection value obtained from the projection data 402 of the subject 10 is converted into the transmission length. The conversion expression may be calculated for each channel number ch, or a common conversion expression may be calculated for all the channel numbers ch.

Since a conversion expression that can convert the projection value into the transmission length with high accuracy even in a range in which the projection value is smaller is calculated by the processing flow illustrated in FIG. 3, it is possible to suppress image quality deterioration of the tomographic image generated by the PCCT apparatus.

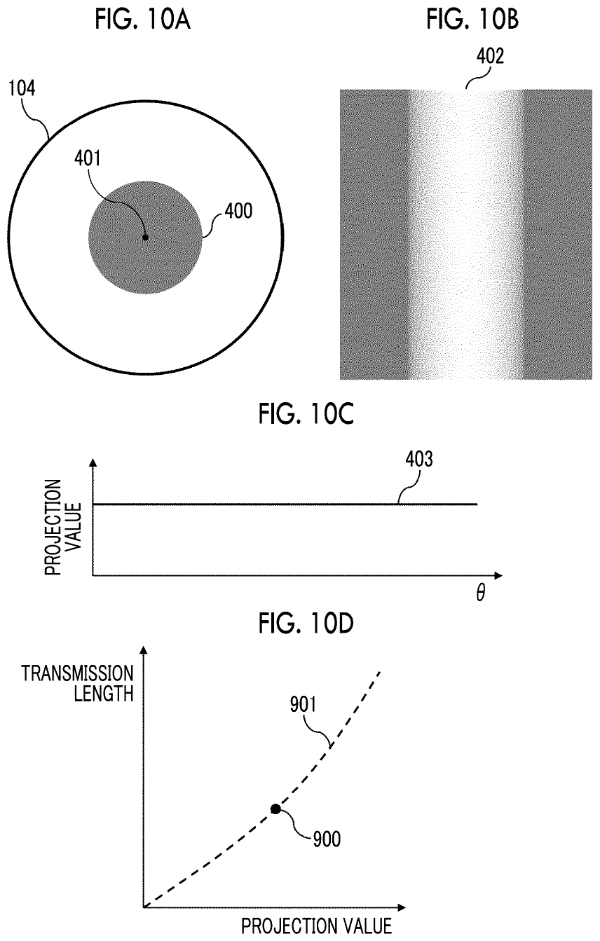

A comparative example with Example I will be described with reference to FIGS. 10A to 10D. In a case where a center of the phantom 400 overlaps the rotation center 401 of the scanner 100 as shown in FIG. 10A, the projection data 402 of the phantom 400 is as shown in FIG. 10B, and the projection profile 403 indicates a constant projection value regardless of the projection angle θ as shown in FIG. 10C. As a result, the measurement point 900 plotted on a coordinate plane in which the projection value is on a horizontal axis and the transmission length is on a vertical axis is only one point as shown in FIG. 10D, and thus the fitting cannot be performed with the approximate curve 901, and the conversion expression for converting the projection value of the subject 10 into the transmission length cannot be calculated.

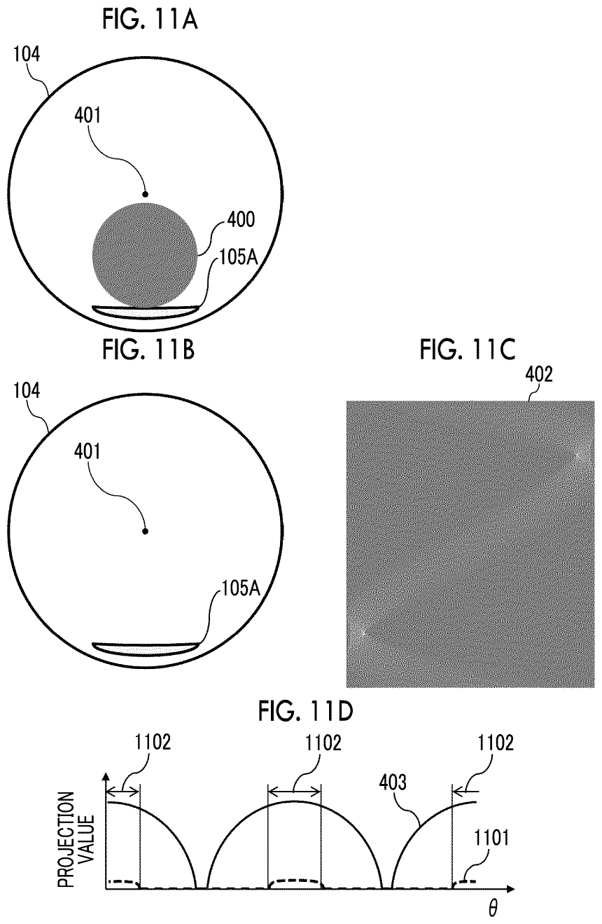

The disposition of the phantom 400 is not limited by the jig 500 illustrated in FIG. 5. A case where the phantom 400 is placed on the top plate 105A of the examination table 105 will be described with reference to FIGS. 11A to 11D. In a case where the phantom 400 is placed on the top plate 105A as shown in FIG. 11A, the projection data 402 of the phantom 400 includes a projection value of the top plate 105A. Therefore, the projection data 402 of the top plate 105A illustrated in FIG. 11C may be acquired by performing rotational imaging in a state in which only the top plate 105A is placed in the opening portion 104 as shown in FIG. 11B, and the acquired projection data 402 may be used to calculate the conversion expression.

For example, a projection profile 1101 of the top plate 105A obtained from the projection data 402 in FIG. 11C overlaps the projection profile 403 of the phantom 400 placed on the top plate 105A to specify a region 1102 including the top plate 105A. Then, the projection value of the specified region 1102 is not used to calculate the conversion expression. In addition, the projection profile 1101 of the top plate 105A may be subtracted from the projection profile 403 of the phantom 400 placed on the top plate 105A to calculate the projection value of only the phantom 400. By using the projection data 402 of the top plate 105A to calculate the conversion expression, it is possible to suppress deterioration in correction accuracy of the conversion expression. In addition, by placing the phantom 400 on the top plate 105A, the jig 500 as shown in FIG. 5 can be made unnecessary.

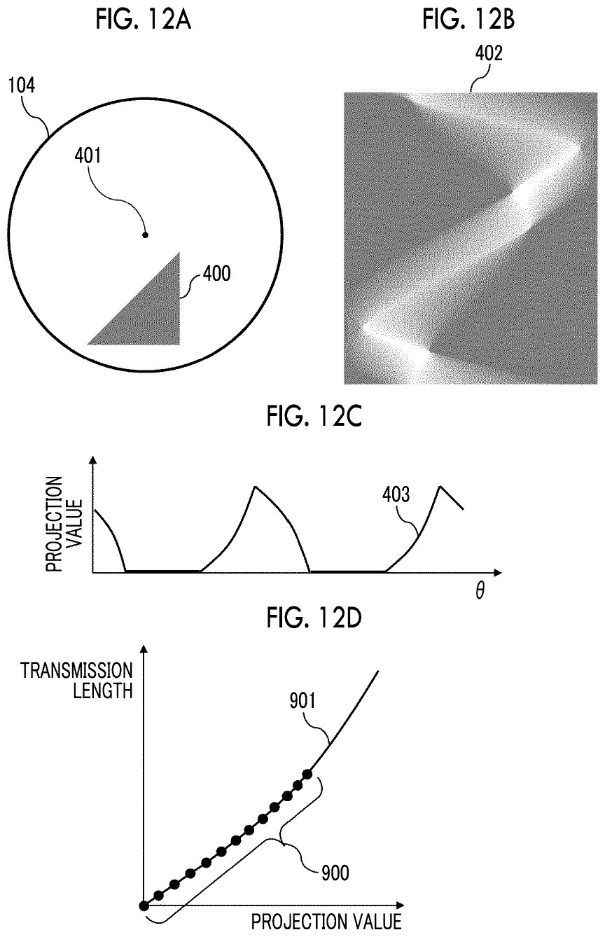

The cross-sectional shape of the phantom 400 is not limited to a circle. A case where the phantom 400 having a polygonal cross-sectional shape is used will be described with reference to FIGS. 12A to 12D. The projection data 402 illustrated in FIG. 12B is acquired by rotational imaging in a state in which the phantom 400 having a right-angled triangular cross-sectional shape as shown in FIG. 12A is disposed not to overlap the rotation center 401. The projection profile 403 obtained from the projection data 402 in FIG. 12B includes a region in which the inclination of the projection value with respect to the projection angle θ is smaller than that in a case of a circular shape illustrated in (c) of FIG. 4, as illustrated in FIG. 12C. As a result, a sampling interval of the projection value is shorter in a range in which the projection value is close to zero, and the measurement points 900 are also densely plotted in a range in which the projection value is close to zero as shown in FIG. 12D. Therefore, the correction accuracy of the projection value in a range in which the projection value is smaller can be further improved. It should be noted that, as the internal angle of the vertex of the polygonal shape disposed in the vicinity of the rotation center 401 is smaller, the sampling interval of the projection value is shorter in a range in which the projection value is close to zero. Therefore, it is preferable that the cross-sectional shape of the phantom 400 is a polygonal shape having an acute internal angle.

The examples of the present invention have been described above. The present invention is not limited to the above examples, and the components can be modified and embodied without departing from the spirit of the invention. In addition, a plurality of the components disclosed in the above examples may be combined as appropriate. Further, some components may be deleted from all the components shown in the above examples.

### EXPLANATION OF REFERENCES

10: subject
100: scanner
101: X-ray source
102: rotating plate
103: collimator
104: opening portion
105: examination table
105A: top plate
105B: base
106: photon counting detector
107: data collection unit
108: rotating plate controller
109: examination table controller
110: X-ray controller
111: high-voltage generation unit
120: operation unit
121: input unit
122: image generation unit
123: storage unit
124: system controller
125: display unit
400: phantom
401: rotation center
402: projection data
403: projection profile
500: jig
700: imaging field of view
701: phantom region
702: ring artifact
801: transmission length
802: transmission length
900: measurement point
901: approximate curve
1101: projection profile
1102: region

What is claimed is:

1. A PCCT apparatus comprising:
a scanner that rotates an X-ray source which irradiates a subject with X-rays and a photon counting detector which detects X-rays transmitted through the subject for each of a plurality of energy bins, around the subject;
an image generation unit that generates a tomographic image by using projection data calculated based on an output of the photon counting detector; and
a controller that controls each unit,
wherein the controller calculates a conversion expression for converting a projection value of the subject into a transmission length by using a plurality of projection values acquired by performing rotational imaging on a phantom which is disposed not to overlap a rotation center of the scanner and of which a material, a shape, and a size are known, along with a plurality of transmission lengths of the phantom.

2. The PCCT apparatus according to claim 1,
wherein a cross-sectional shape of the phantom is a polygonal shape having an acute internal angle.

3. The PCCT apparatus according to claim 2,
wherein a vertex of the acute internal angle of the phantom is disposed in a vicinity of the rotation center of the scanner.

4. The PCCT apparatus according to claim 1,
wherein, in a case where the phantom is placed on a top plate, a projection value including the top plate is not used to calculate the conversion expression.

5. The PCCT apparatus according to claim 1,
wherein the controller calculates a position of the phantom by using projection data of the phantom, prior to obtaining the transmission length of the phantom.

6. The PCCT apparatus according to claim 1,
wherein the controller calculates a position of the phantom by using a tomographic image generated from projection data of the phantom, prior to obtaining the transmission length of the phantom.

7. A control method of a PCCT apparatus including a scanner that rotates an X-ray source which irradiates a subject with X-rays and a photon counting detector which detects X-rays transmitted through the subject for each of a plurality of energy bins, around the subject, and an image generation unit that generates a tomographic image by using projection data calculated based on an output of the photon counting detector, the control method comprising:
calculating a conversion expression for converting a projection value of the subject into a transmission length by using a plurality of projection values acquired by performing rotational imaging on a phantom which is disposed not to overlap a rotation center of the scanner and of which a material, a shape, and a size are known, along with a plurality of transmission lengths of the phantom.
